(19) United States
(12) Patent Application Publication
(10) Pub. No.: US 2022/0359068 A1
(43) Pub. Date: Nov. 10, 2022

(54) MOBILE MEDICAL SYSTEM AND METHOD

(71) Applicant: Rodney Abdulkarim, Los Angeles, CA (US)

(72) Inventor: Rodney Abdulkarim, Los Angeles, CA (US)

(73) Assignee: Propel Technology Services, Inc., ONTARIO, CA (US)

(21) Appl. No.: 17/313,927

(22) Filed: May 6, 2021

(51) Int. Cl. G16H 40/67; G16H 50/20; G06Q 10/04; G06Q 10/06; G06Q 30/02

(52) U.S. Cl. CPC G16H 40/67; G16H 50/20; G06Q 10/047; G06Q 10/06311; G06Q 30/0284

(57) ABSTRACT

A system that automates and optimizes the workflows and transportation routes of one or more mobile medical units is provided. The mobile medical units may include mobile phlebotomy units for the onsite collection of medical specimens (e.g., blood). The system may include a plurality of mobile medical units (e.g., medically equipped vans) within a particular city, and a system for receiving and assigning medical workorders to particular mobile units (e.g., based on the mobile unit's location compared to that of the patient, availability, and other criteria). The system also may determine an optimized route to the patient for the mobile unit to follow and may provide additional administrative support to the mobile unit during the entire workflow process. The system also may perform onsite diagnostic tests on medical specimens collected from a patient (e.g., using medical test equipment), may flag abnormal test results, and may communicate the results to a healthcare provider. The system also may effect a particular treatment or prophylaxis for a disease or medical condition of a patient as indicated by the diagnostic test results acquired by a mobile medical unit at the patient's remote location.

### FIELD OF THE INVENTION

This invention relates to a framework, system, and method of mobile medical systems, including mobile medical systems for phlebotomy procedures.

### BACKGROUND

Mobile medical units that provide medical treatments and tests at the residence of a patient are becoming popular throughout the world. Because the mobile units may bring all of the necessary medical test equipment to the patient's location, the patient is not required to travel and/or otherwise leave his/her home. This may be extremely helpful to elderly patients and/or to patients with disabilities that make it difficult for them to travel. For example, a patient that requires a phlebotomy procedure may have the blood specimens drawn at his/her home, have the specimen analyzed in onsite, and be provided the test results in real time.

However, the provision of mobile medical units adds additional complexity to the overall process of retrieving medical specimens (e.g., blood samples), testing the specimens, and then providing the test results and analyses to the healthcare providers. For example, the mobile units require a dispatch service that may assign workorders to the units, a process to track the locations of the units, a process to assign appropriate units to particular locations, a process to communicate the medical test results from offsite locations to the appropriate healthcare providers, and the administrative support that is required for each step.

Accordingly, there is a need for a system and method to provide mobile medical systems that addresses and solves at least the complexities described above. There also is a need for a mobile medical system that effects a particular treatment or prophylaxis for a disease or medical condition indicated by the diagnostic test results acquired by a mobile medical unit at a patient's remote location.

### BRIEF DESCRIPTION OF THE DRAWINGS

Other objects, features, and characteristics of the present invention as well as the methods of operation and functions of the related elements of structure, and the combination of parts and economies of manufacture, will become more apparent upon consideration of the following description and the appended claims with reference to the accompanying drawings, all of which form a part of this specification. None of the drawings are to scale unless specifically stated otherwise.

FIG. 1 shows an overview of a mobile medical system framework in accordance with exemplary embodiments hereof;

FIG. 2 shows actions taken a mobile medical system in accordance with exemplary embodiments hereof;

FIG. 3 shows example GUIs and dashboards of a mobile medical system in accordance with exemplary embodiments hereof;

FIGS. 4-5 show example data reports generated by a mobile medical system in accordance with exemplary embodiments hereof;

FIG. 6 shows an example email provided by and dashboards of a mobile medical system in accordance with exemplary embodiments hereof;

FIG. 7 shows an example mobile application GUI provided by a mobile medical system in accordance with exemplary embodiments hereof;

FIG. 8 shows aspects of a mobile medical system in accordance with exemplary embodiments hereof; and

FIG. 9 depicts aspects of computing and computer devices in accordance with exemplary embodiments hereof.

### DETAILED DESCRIPTION OF EXEMPLARY EMBODIMENTS

As used herein, unless used otherwise, the following terms and abbreviations have the following meanings:

As is known in the art, phlebotomy refers to the process of making a puncture in a vein, usually in the arm, for the purpose of drawing blood. The procedure also may be referred to as venipuncture. Phlebotomy typically utilizes an Evacuated Tube System (ETS) to perform the procedure comprising a needle, a tube holder, and one or more evacuated tubes.

The term “patient” will refer to a person requiring a medical procedure, such as, without limitation, a phlebotomy procedure. A patient may include any type of person.

The term “technician” (as in “phlebotomy technician” or “medical technician”) will refer to a person performing a medical procedure, such as, without limitation, a phlebotomy procedure on the patient. For example, the technician may include medical laboratory scientists, medical practitioners, emergency medical technicians (EMTs), paramedics, phlebotomists, dialysis technicians, nurses, medical doctors, other types of technicians, and any combinations thereof. It is understood that while phlebotomy is typically performed on humans, it also may be performed on animals by veterinarians or veterinary technicians (at which time the patient may include an animal).

The term “client” will refer to a person or entity requesting a medical procedure to be performed on a patient by a technician. For example, a client may include a medical doctor, a nurse, or other medical professional, a medical office, a hospital, a medical insurance provider, any other type of client and any combinations thereof.

The term “administrator” will refer to a user of the system responsible for administrative and other types of system-related activities. In general, an administrator may be located in a central location and may provide work-related support to one or more mobile technicians during use of the system.

In general, the system according to exemplary embodiments hereof provides a cloud-based software platform that automates and optimizes the workflows and transportation routes of one or more mobile medical units. In some embodiments, the mobile medical units may be deployed as mobile phlebotomy units and/or as other types of clinical medical laboratories for the onsite collection of medical specimens (e.g., blood).

For example, in some embodiments, the system may include a plurality of mobile medical units (e.g., medically equipped vans) within a particular city, and when the system may receive a workorder for a particular patient located at a particular location within the city, the system may determine which mobile medical unit to send to the patient to perform the procedure (e.g., based on the mobile unit's location compared to that of the patient, availability, and other criteria). The system also may determine an optimized route to the patient for the mobile unit to follow and may provide additional administrative support to the mobile unit during the entire workflow process. The system also may perform onsite diagnostic tests on medical specimens collected from a patient, may flag abnormal test results and may communicate the results to a healthcare provider. This will be described in detail in other sections.

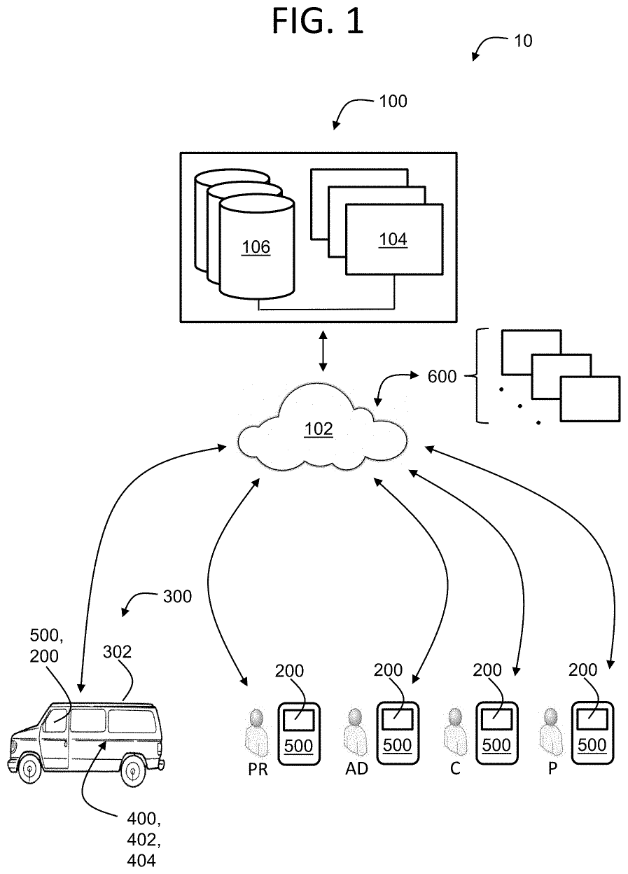

FIG. 1 shows an overview of an exemplary framework for a mobile medical system 10 (also referred to simply as the system 10) according to exemplary embodiments hereof. As shown, the mobile medical system 10 may include a backend system 100 that may interface with an application 200 (e.g., a mobile application or “app”, a website viewed using a browser, a desktop application, or other types of applications), a transportation system 300 including one or more mobile units 302, and one or more medical apparatuses 400 (e.g., carried within the mobile units 302). The interface may include a network 102 (e.g., the Internet, LAN, WAN, etc.), wireless communication systems, cellular communication systems, telephony or other types of communication systems or protocols.

The system 10 may be accessed by multiple users (e.g., one or more technician(s) T, one or more administrator(s) AD, one or more client(s) C, and/or one or more patient(s) P) via the network 102 and using the mobile application 200 running on one or more computing devices 500 (e.g., smart phones, tablet computers, laptops, desktop computers, mobile media players, other types of client devices, etc.). While FIG. 1 depicts one mobile unit 302, one technician T, one administrator AD, one client C, and one patient P, it is understood that the system 10 may include any number of mobile units 302, technicians T, administrators AD, clients C, and/or patients P.

In some embodiments, the system 10 also may interface with one or more external systems 600 such as, without limitation, laboratory information systems (LIS), electronic medical record systems (EMR), healthcare providers, medical offices, hospitals, medical insurance providers, auditors, etc.). In some embodiments, the external systems 600 may include one or more clients C.

In some embodiments, the application 200 may display one or more graphical user interfaces (GUIs) on the display(s) of associated electronic devices 500. In some embodiments, the application 200 may provide different GUIs to different types of users depending on the functionalities required by each respective user. For example, the application 200 may provide a technician T with a first set of GUIs tailored to the workflows that the technician T may perform during his/her daily routine (as described herein), a second set of GUIs to an administrator AD tailored to the workflows that the administrator AD may be required to perform (e.g., receiving workorders WO from clients C, assigning workorders WO to technicians T, tracking the status of workorders WO, etc.), and a third set of GUIs to a client C tailored to the workflows that the client C may be required to perform (e.g., tracking the status of workorders WO, receiving and viewing diagnostic data taken by the system 10 pertaining to various workorders WO and/or patients P, etc.). It is understood that some GUIs may be provided to several types of users T, AD, C, and that other GUIs may be provided to one type or another. It also is understood that some GUIs may be provided through a mobile application 200 running on a personal electronics device 500, through a website portal or dashboard 200, through a desktop application 200, and/or through other types of applications 200, depending on the user.

In some embodiments, the backend system 100 may include a cloud platform (e.g., one or more backend servers), one or more local controllers, or any combination thereof. In some embodiments, the backend system 100 includes a cloud platform that interfaces with one or more local controllers. For example, administrators of the system 10 may interface with the system 10 via a local controller in communication with a cloud platform.

The backend system 100 may include one or more applications 104 and one or more databases 106 as will be described in other sections.

In some embodiments, the transportation system 300 includes one or more mobile units 302. The mobile units 302 may include any type of vehicle configured with the system 10 that may transport at least one technician T to the location of at least one patient P. For example, a mobile unit 302 may include (without limitation) a van, a truck, an automobile, a trailer, a motorcycle, a bicycle, a helicopter, a plane, a boat, a train, any other type of vehicle, and any combinations thereof.

In some embodiments, the system 10 (e.g., the transportation system 300) includes a geolocation and navigation system (e.g.; a Global Positioning System (GPS)) enabled to track the real time location of the one or more mobile units 302 and/or the electronic devices 500 associated with the mobile units 302 and/or the technicians T. For example, in some embodiments, a mobile unit 302 may include an integrated device 500 (running the application 200) whose location may be tracked by the system 10. In another example, a technician T may utilize a personal device 500 (running the application 200) whose location may be tracked by the system 10. It is understood that the system 10 may be configured to track the location of any device 500 associated and/or configured with the system 10.

In some embodiments, the mobile units 302 may be equipped with one or more medical apparatuses 400 as required to perform one or more medical procedures (e.g., phlebotomy procedures). In some embodiments, the medical apparatuses 400 may include medical equipment 402 such as (without limitation) Evacuated Tube Systems (ETS), Point of Care (POC) diagnostic devices, meters, and other types of medical equipment. In some embodiments the POC devices may be used to obtain diagnostic results while the technician T is with (or close to) the patient P. For example, the POC devices may be used to perform blood glucose testing, blood gas and electrolytes analysis, rapid coagulation testing, rapid cardiac markers diagnostics, cholesterol screening, drugs of abuse screening, urine strips testing, pregnancy testing, fecal occult blood analysis, food pathogens screening, hemoglobin diagnostics, infectious disease testing (such as COVID-19 rapid tests), and other types of diagnostics.

In some embodiments, the POC tests include a prothrombin time (PT) test that measures the amount of time it takes for a clot to form in a blood sample after reagents are added. The PT is often performed in parallel with a partial thromboplastin time (PTT) test and together the tests may assess the levels and proper functioning of coagulation factors within the blood. Using this information, the system 10 also may calculate the Internationalized Normalized Ratio (INR) as a standardized result.

In some embodiments, the system 10 interfaces with a PT test meter 402, a PTT test meter 402, and/or an INR test meter 402 to perform the tests. The system 10 may control the test meters 402 (using drivers) and/or may collect the test results for diagnostic purposes. The diagnostics may be performed within the test meters 402 and/or by the system 10 itself (e.g., on its backend 100). In general, the system 10 and/or the test meters 402 reads a test strip (containing a blood sample and added reagents), measures how long it takes the blood to clot, and provides the results as PT results and/or as results based on the standardized calculation in the form of the International Normalized Ratio, or INR. In some embodiments, the system 10 interfaces with the PT test meter 402, the PTT test meter 402, and/or the INR test meter 402 using a wireless communication protocol (e.g., Wi-Fi, Bluetooth, etc.), a communications cable, and/or by other communication methods.

In other embodiments, the apparatuses 400 may include ancillary equipment 404 such as (without limitation) portable containers to safely house and store the medical equipment 402 (e.g., containers for the un-used and used EFTs, etc.), medical stations including stands that hold and/or facilitate the use of the medical equipment 402, accessories to assist the patient P during the procedures such as medical chairs, beds, barcode and/or label printers, and/or other accessories. In this way, a mobile unit 302 may transport a technician T and the necessary medical apparatuses 400 to the location of a patient P to perform the required procedures.

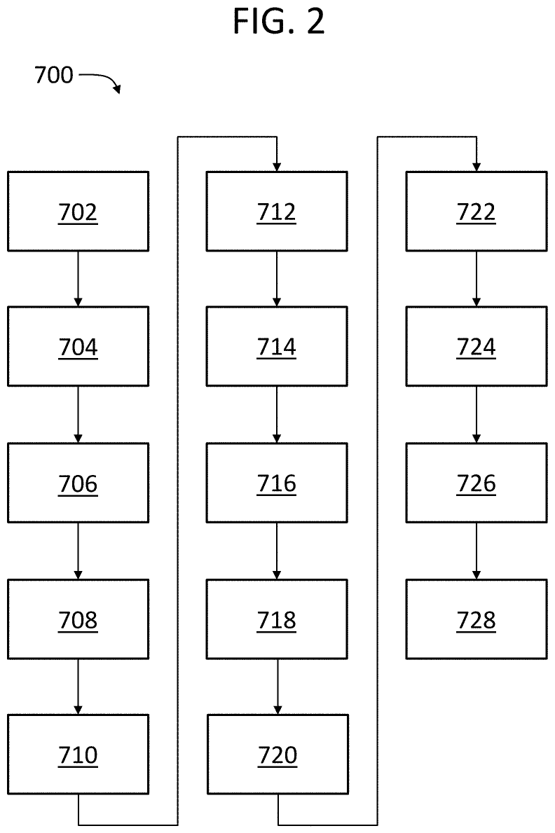

FIG. 2 shows an overview of workflow actions 700 that the system 10 may perform during use. Further details of these actions will be described in other sections. In some embodiments, the system 10 may perform one or more of the following actions:
702: Electronically receive one or more laboratory phlebotomy workorders WO (e.g., from a client C) and assign the workorders WO to specific mobile units 302 to fulfill onsite (at the location of each respective patient P). The workorders WO may be received from an external system 600 and/or client C such as a healthcare provider;
704: For each workorder WO received, determine the optimal mobile unit 302 (and associated technician T) to assign to the workorder WO. This may be based on location of the mobile units 302 and that of the patient P, current workloads of each mobile unit 302 in the field, available staff, batched orders, technician expertise, medical apparatuses 400 availability with particular mobile units 302, and other criteria;
706: Provide special handling and scheduling instructions for workorders WO that require special conditions. For example, the system 10 may identify workorder procedures that require the patient P to fast prior to the visit as well as any medically urgent blood draws. In these cases, the system 10 provides decision support logic to prioritize and distribute the workorders WO to particular mobile units 302 and/or technicians T best suited to handle the special conditions. Criteria such as that described above as well as other criteria may be used when making the determinations. In this way, the system 10 may minimize any delay of care regarding orders with medical special handling requirements.
708: Once a workorder WO is assigned to a mobile unit 302, determine an optimal transportation route in real time for the mobile unit 302 to take from its current location to that of the respective patient P. In some embodiments, this may include displaying the route on an interactive map via the application 200 running on the mobile unit's electronic device 500). In some embodiments, the system 10 may continuously monitor and adjust the proposed route in real time depending on workflow conditions (e.g., traffic, etc.).
710: Determine the required medical apparatuses 400 and/or medical equipment 402 needed for the particular medial procedures to be performed for a particular workorder WO and patient P. For example, the system 10 may determine which test tubes and POC devices are to be used for a particular phlebotomy procedure and may convey this information to the technician T to use.
712: Perform onsite diagnostic tests (e.g., using Point of Care (POC) devices and test meters 402) on the collected medical specimens (e.g., blood specimens) and provide the results to the administrators AD, the clients C, and/or other entities.
714: Analyze the test results from 712 to determine results that are within a defined normal diagnostic range(s) and/or results that are outside a defined normal diagnostic range(s). For those results deemed to be outside normal diagnostic range(s), flag these results as “Critical Results” and notify the appropriate administrators AD, clients C, and/or other entities accordingly so that appropriate actions may be taken.
716: Determine if all required procedures were accomplished during the visit, and it not, communicate this to the appropriate entities and schedule a return visit. For example, if a urine specimen was not available at the time of the visit during which blood specimens were collected, the system 10 may automatically trigger a notification to the administrators AD and/or the client C and create a return visit order to be scheduled.
718: Provide a secure data exchange platform for all workflows, communications, and data transfers between the backend system 100 and each mobile unit 302 and technician T during the fulfillment of each workorder WO;
720: Provide real time communication between the mobile units 302, the technicians T, the administrators AD, the clients C, the patients P, and any other associated entities (e.g., a central office or laboratory);
722: Administratively manage and document all workflows and data transfers;
724: Interface with clients C and other external systems 600 such as laboratory information systems (LIS), electronic medical record systems (EMR), healthcare providers, etc.;
726: Calculate travel charges associated with the fulfillment of each workorder WO as appropriate for billing purposes; and
728: Provide other services.

It is understood that the actions described above are meant for demonstration and are non-limiting, and that the system 10 may perform other actions not described, and/or not all of the actions 700 listed. It is also understood that the system 10 may perform the actions 700 in other orders.

Additional embodiments and details of the system 10 will be described by way of several detailed use scenarios. The use scenarios provided below are chosen to illustrate various embodiments and implementations of the system 10, and those of ordinary skill in the art will appreciate and understand, upon reading this description, that the examples are not limiting and that the system 10 may be used in different ways. It also is understood that details and elements of different embodiments described in different scenarios and examples may be combined in any way to form additional embodiments all of which are within the scope of the system 10.

In a first example, the system 10 may electronically receive a first workorder WO1 from a client C for a phlebotomy procedure to be performed on a first patient P1 located at a first location L1. The system 10 may receive the order WO1 through its backend system 100 via an application 200 running on one or more electronic devices 500. For example, an administrator AD may receive the workorder WO1 on a desktop computer 500 at a central office. The workorder WO1 may originate from a client's LIS or from other systems interfacing with the system 10 and/or a client C. Once received, the system 10 may check to ensure that the workorder WO1 is not a duplicate order while enabling the administrator AD to confirm the order WO1, to add notes and/or updates to the order WO1, to edit the order WO1 as necessary, and to perform other activities regarding the order WO1.

Next (e.g., after the workorder WO1 is confirmed and/or after a cutoff time has been reached), the system 10 may authorize the workorder WO1 procedure and may assign the workorder WO1 to a mobile unit 302 and associated technician T to fulfill. Once assigned the workorder WO1, the mobile unit 302 and its associated technician T may travel to the location of the patient P1 and perform the phlebotomy procedure onsite.

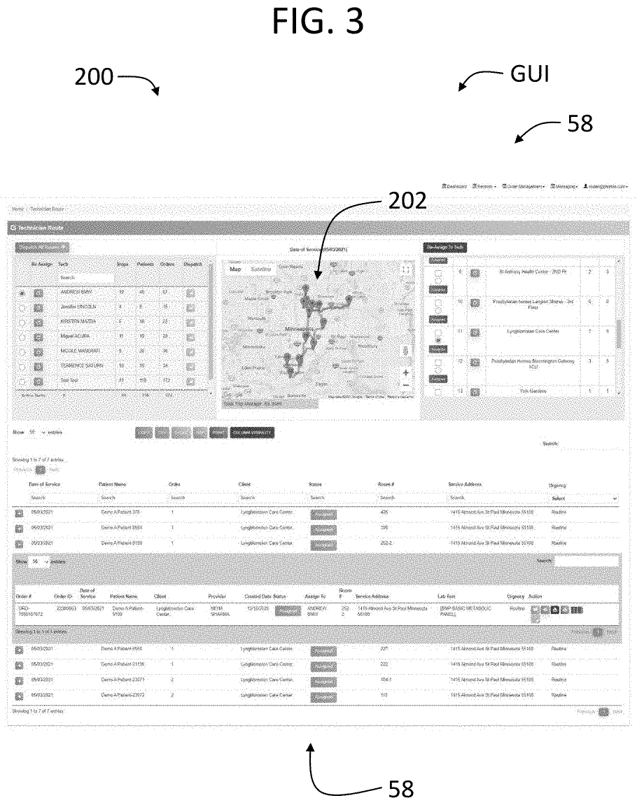

In some embodiments, the system 10 (e.g., the transportation system 300) may determine an optimal transportation route in real time for the assigned mobile unit 302 to follow from its current location to that of the respective patient P1. In some embodiments, this may include displaying the route on an interactive map 202 on a GUI via the application 200 running on the mobile unit's electronic device 500) as shown in FIG. 3. In addition, the transportation system 300 may continually monitor the location of the mobile unit 302 in real time enroute to the patient P1 and depending on various identified real time conditions (e.g., traffic, road work, detours, speed of the mobile unit 302, etc.), the system 10 may reroute the mobile unit 302 to take a modified route to the patient P1 instead. This functionality also may be implemented through an interactive map via the application 200 as described above.

In some embodiments, the system 10 may update the patient P with information regarding the scheduled appointment. For example, the system 10 may send the patient P a reminder of the appointment (e.g., via email the day prior), information regarding the appointment (e.g., whether or not the patient P needs to fast, etc.), when the mobile unit 302 and technician T has been dispatched in real time, the expected time that the mobile unit 302 and technician T will arrive to the patient's location, and any other appropriate information. Once the mobile unit 302 and technician T arrive at the patient's location, the system 10 may call the patient P to inform him/her of this fact and to come meet the unit 302 (e.g., outside the patient's home and/or at the mobile unit 302).

In some embodiments, when more than one mobile unit 302 and associated technician T is available (e.g., each at different locations), the system 10 may determine a specific mobile unit 302 and technician T to receive and fulfill the workorder WO1. In some embodiments, the system 10 may apply one or more criteria to make this determination, including, without limitation, (i) the location of each mobile unit 302 with respect to the location of the patient P1 and/or to system service hubs, N the current workloads of each mobile unit 302 in the field, (iii) available technicians T associated with each mobile unit 302, (iv) the work schedules of the available technicians T, (v) other workorders WO assigned to technicians T and associated with other patients P within the vicinity of the patient P1, (vi) batched workorders WO assigned to particular mobile units 302, (vii) time of day, and (viii) other criteria. Once the workorder WO has been assigned, the system 10 may determine the optimal route for the mobile unit 302 to take as described above.

At any point in time, the system 10 may enable an administrator AD to review the location of each available mobile unit 302 and associated technicians T, as well as the workorder assignment(s) and adjust them, as necessary. For example, an administrator may wish to reassign a particular workorder WO from one mobile unit 302 to a different mobile unit 302 (e.g., due to traffic conditions, cancelled appointments, etc.) and may do so using the application 200. The administrator AD also may wish to alter the suggested route(s) assigned to any mobile unit 302 and may do so via the application 200 as well. It is understood that the system 10 may enable an administrator AD to make any modifications as necessary regarding the workorders WO, mobile units 302, technicians T, patients P, and other elements associated with the system 10 and its use.

Once the system 10 has assigned a particular workorder WO to a particular mobile unit 302 and associated technician T, the system 10 may notify technician T accordingly. The system 10 may provide information relating to the workorder 10 the technician T via the application 200. For example, the system 10 may provide to the technician T the patient's contact information (e.g., name, account number, location, phone number, email address, etc.), the time window of appointment, procedures to be performed, necessary preparations for the procedure(s), and other appropriate information.

In some embodiments, once a technician T receives a workorder assignment, he/she may accept or reject the assignment. If the technician T rejects the assignment, he/she may enter notes into the system 10 (e.g., via the application 200) to provide the reasons for the rejection. If this occurs, the system 10 may revoke the assignment and determine a different mobile unit 302 and associated technician T to assign the workorder WO. During this procedure, the system 10 may use the same or similar methodologies, criteria, and workflow operations as described above.

Once a mobile unit 302 and associated technician T has arrived at the patient's location, the technician T may check-in the patient P by collecting pertinent patient information and inputting it to the system 10 (e.g., via the application 200 running on his/her device 500).

In some embodiments, the system 10 determines the required medical apparatuses 400 needed to perform the procedures required by the workorder WO and informs the responsible technician T of this information.

In some embodiments, the system 10 provides software wizards or other types of guidance on the GUI of the application 200 to the technician T for each procedure to be performed. In this way, the system 10 may guide the technician(s) T during each procedure.

The technician T may then perform the medical procedure (e.g., the phlebotomy procedure). Then, once the procedure is completed and the medical specimen (e.g., a blood sample) has been collected (e.g., into an evacuated tube), the system 10 may generate an identification label for the sample, may print the label using a printer (e.g., a mobile Bluetooth printer), and the technician T may place the label onto the respective sample (e.g., onto the evacuated tube). In some embodiments, the identification label may include the patient's name, birthdate, address, phone number, account number, other information such as the appointment date and the technician's identifying information, and any combinations thereof. The label also may include a barcode, a QR code, and/or any other types of alpha, numeric and/or graphical codes for identification of the tube.

In some embodiments, the system 10 interfaces with and/or controls the POC devices 400 to perform diagnostic tests on the medical samples taken by the technician T. The system 10 may then collect the data from the devices 400, format the data into test reports, and communicate the reports to appropriate entities (e.g., an administrator AD, a client C, etc.). In some embodiments, the system 10 analyzes the test results and flags results that are outside a defined normal diagnostic range. For example, the system 10 may flag such results as “Critical Results” on a test report that is provided to a client C such that the client C may quickly recognize the abnormal data and take the steps necessary to provide appropriate healthcare to the patient P.

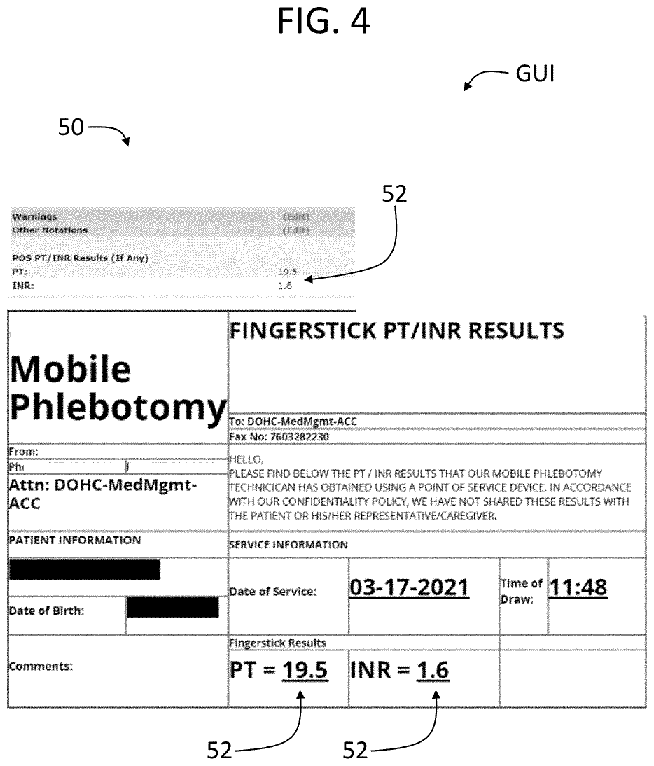

In one example as shown in FIG. 4, the system 10 may take and/or acquire the diagnostic test results and format the results into a report 50 or other type of datasheet. In some embodiments, the report 50 may be displayed on the GUI of the application 200, and in other embodiments, the report 50 may be communicated (e.g., via email over the network 102) to the appropriate entities such as an administrator AD, a client C, a patient P, and/or other entities. In other embodiments, the report 50 may be provided as a hardcopy (e.g., printed on a sheet of paper), in a PDF format that may be viewed on a display and/or printed, or as other suitable formats. FIG. 4 shows a sample report 50 indicating diagnostic test results 52 that are deemed to fall within a defined normal diagnostic range.

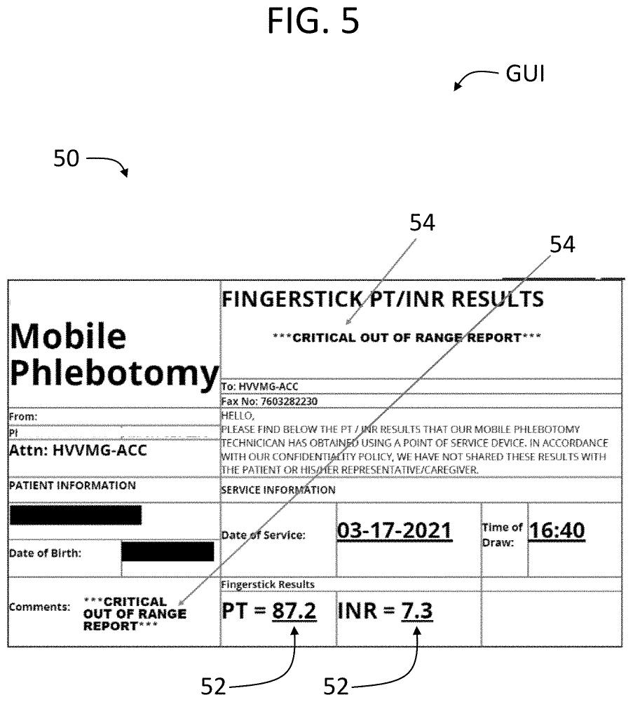

In another example as shown in FIG. 5, the system 10 may analyze and deem diagnostic results to be outside defined normal diagnostic values, and accordingly, may provide the test results 52 on a report 50 while providing a warning alert 54 that the results are abnormal. In this instance, the system 10 also may provide and email 56 (or other type of communication) to an administrator AD, a client C, or other entity indicating that the results are outside normal values. This scenario, the system 10 also may provide information and/or guidance to the client C (e.g., to the healthcare provider of the patient P) to effect a particular treatment or prophylaxis for a disease or medical condition indicated by the abnormal diagnostic test results.

an additional element that applies or uses a judicial exception to effect a particular treatment or prophylaxis for a disease or medical condition.

For example, in some embodiments when the system 10 analyzes and deems that a patient's PT, PTT, and/or INR test results are outside normal diagnostic range(s), the system 10 may recommend appropriate action(s) to be taken by the patient P and/or the healthcare provider C to tailor a medical treatment for the patient P based at least in part on the diagnostic data and the analysis of the data performed by the system 10. In one example, the system 10 may recommend the healthcare provider C to prescribe a particular medication to the patient P (e.g., warfarin) to bring the patient's diagnostic test results back into a normal range. In another example, the system 10 may recommend that the dosage of a particular medication be modified given the newly taken test results. In another example, that the patient P increase his/her intake of vitamin K and may suggest foods that provide suitable levels of vitamin K as part of a healthy diet. It is understood that the system 10 may effect a particular treatment or prophylaxis for a disease or medical condition of a patient P by performing any type of appropriate medical tests, by analyzing the subsequent test results, by determining diagnostic results that are outside defined normal diagnostic values, by providing the test results to the patient P and/or the client C, by highlighting the abnormal results, by providing guidance and information regarding new and/or modified healthcare treatment plans for the patient P based on the test results and the analysis, and by any combination thereof.

In some embodiments, the system 10 also may provide a dashboard 58 on a system GUI accessible by an administrator AD, a client C, or other entities, indicating the test results and statuses (also see the dashboards 58 shown in FIG. 3 displaying workorder WO listings, workorder WO assignments and/or reassignments to technicians T, etc., that may be utilized by an administrator AD, for example). In some embodiments, test results deemed abnormal may be color coded (e.g., red) and/or as bold or all caps to indicate that a problem may exist.

In some embodiments, the system 10 may communicate information relating to the performed procedure (e.g., the information described above) to the backend system 100, to the administrators AD, and/or to other entities as required. In this way, the system 10 may notify each interested party that the workorder WO has been completed. In some embodiments, applicable communications may utilize secure HL7 data exchange methods and/or other secure means of communication as appropriate.

It is understood that as the system 10 receives additional workorders WO for additional patients P, that the system 10 may perform the activities described above for each workorder received. For example, the system 10 may determine which mobile unit 302 and associated technician T to assign to the workorder WO based on the locations of the mobile units 302 (current locations and/or future scheduled locations), other workorders WO assigned to mobile units 302 with patient locations within close proximity (e.g., 5 miles) to the new patient location, current workloads of the mobile units 302 and technicians T, and other criteria.

In some embodiments, the system 10 may receive and batch multiple workorders WO together using criteria such as, without limitation, geographical location of the orders' respective patients P, types of procedures required for each order WO, scheduled appointment time of each order WO, scheduled location of the mobile units 302 and associated technicians T, and other criteria. In this way, workorders WO in close proximity to one another (e.g., within 10 miles) and/or at closely scheduled times (e.g., within the same or similar time window) may be grouped together and assigned to a mobile unit 302 accordingly.

Once a batch of orders WO has been compiled, the batch may be assigned to a particular mobile unit 302 and associated technician T based on the scheduled location of the mobile unit 302, the medical procedures that the unit 302 is equipped to perform, the availability and workload of the mobile unit 302, and other criteria. In this way, a technician T may be assigned a batch of workorders WO and may view his/her schedule and associated transportation routes (e.g., as determined by the transportation system 300 as described above) for his/her shift (or portion thereof). Note that the system 10 may modify the batched orders (e.g., may add workorders WO to or remove workorders WO from) as required throughout the day or shift. In this case, the system 10 may notify each respective mobile unit 302 and associated technician T of the changes via the application 200 (e.g., a GUI displaying a scheduled patient list may be modified, an interactive map may update to show the new schedule of transportation routes and patient locations, etc.).

In some embodiments, the system 10 (e.g., the transportation system 300) may track the distances traveled by each mobile unit 302 enroute to each patient P and determine appropriate travel charges (e.g., charge per mile) to apply to each workorder WO. These travel charges also may be added to the applicable billing documents (e.g., invoices).

In some embodiments, the system 10 includes an internal communications system that enables technicians T, administrators AD, clients C, and/or patients P to communicate with one another in any combinations in real time via email, text, voice, phone, messaging, etc. The communications system also enables technicians T, administrators AD and clients C to securely send files (e.g., documents) to one another.

In some embodiments, the system 10 tracks each relevant action taken by the technicians T, the mobile units 302, the administrators AD, the external entities 600, the patients P, and any other entities or systems associated with the system 10 in real time throughout the workflows. For example, the system 10 may track each action taken from the receipt of the workorders WO and the deployment of the mobile units 302, to the completion of the associated medical procedures and the subsequent billing of the clients (e.g., the external systems 600).

Application 200

In some embodiments, the application 200 resides an electronic device 500 such as a smartphone, a tablet computer, a laptop computer, a mobile music player, other types of electronic devices and any combination thereof. The application 200 includes a graphical user interface (GUI) that may be presented on the device's display and that includes controls (e.g., touchscreen and/or mechanical buttons, etc.) that a technician T, an administrator AD and/or a patient P may activate to interact with the system 10. For example, the GUI may include controls and/or other mechanisms that enable the users T, AD, C, P to interface with the system 10 during its general usage. Other types of controls may include buttons, dials, check boxes, drop-down menus, scroll bars, other types of controls and any combination thereof.

In some embodiments, the application 200 includes voice recognition capabilities so that it may receive and implement voice commands from the users T, AD, C, P. In addition, the application 200 may accommodate any language.

In some embodiments, the application 200 may present instructions, wizards, and/or other types of guidance to the users T, AD, C, P via the GUIs.

An example mobile app GUI presented to a technician T via the application 200 running on the technician's electronic device 500 is shown in FIG. 4.

System Structure

FIG. 5 shows aspects of an exemplary transportation dating system 10 of FIG. 1. As shown, the system 10 and backend system 100 comprises various internal applications 104 and one or more databases 106, described in greater detail below. The internal applications 104 may generally interact with the one or more databases 106 and the data stored therein.

The database(s) 106 may comprise one or more separate or integrated databases, at least some of which may be distributed. The database(s) 106 may be implemented in any manner, and, when made up of more than one database, the various databases need not all be implemented in the same way. It should be appreciated that the system is not limited by the nature or location of database(s) 106 or by the manner in which they are implemented.

Each of the internal applications 104 may provide one or more services via an appropriate interface. Although shown as separate applications 104 for the sake of this description, it is appreciated that some or all of the various applications 104 may be combined. The various applications 104 may be implemented in any manner and need not all be implemented in the same way (e.g., using the same software languages, interfaces, or protocols).

In some embodiments, the applications 104 may include one or more of the following applications 104:
1. Data input application(s) 108. This application may input any type of data from any applicable system and/or element such as the application 200, medical apparatuses 400 (e.g., a POC device), external entities 600, any other system and/or element and any combination thereof.
2. Data output application(s) 110. This application may output any type of data to any applicable system and/or element such as the application 200, external entities 600, any other system and/or element and any combination thereof.
3. Transportation system application(s) 112. This application may include any and all of the applications necessary for the transportation system 300 to perform its functionalities. This application includes a Global Positioning System (GPS) or other types of geolocation systems to locate and track the position of the mobile units 302, the location(s) of the patients P, and any other types of locations as required by the system 10.
4. Data reporting application(s) 114. This application may generate any type of report regarding the use and/or functionalities of the system 10 including completed workorders WO, diagnostic tests, etc.
5. Communications applications 116. This application provides the functionalities needed for the various parties associated with the system 10 (e.g., the technicians T, the administrators AD, the clients C, etc.) to communicate with one another (e.g., via phone, text, email, messaging, etc.).
6. Miscellaneous applications 118. These applications will provide the functionalities of all addition workflow requirements of the system 10.

The applications 104 also may include other applications and/or auxiliary applications (not shown). Those of ordinary skill in the art will appreciate and understand, upon reading this description, that the above list of applications is meant for demonstration and that the system 10 may include other applications that may be necessary for the system 10 to generally perform its functionalities as described in this specification. In addition, as should be appreciated, embodiments or implementations of the system 10 need not include all of the applications listed, and that some or all of the applications may be optional. It is also understood that the scope of the system 10 is not limited in any way by the applications that it may include.

In some embodiments, the database(s) 106 may include one or more of the following databases 106:
1. Patient information database(s) 120. This database may store any registration and/or profile information for any patient P associated with any workorder WO.
2. Mobile unit and technician database(s) 122. This database may store information regarding each mobile unit 302 (e.g., identification, equipment it may carry, schedule, etc.) and each associated technician T (e.g., identification, authorizations, expertise, schedule, workload, etc.).
3. Usage history database(s) 124. This database may store any type of historical data pertaining to any workorder WO, patient P, technician T, administrator AD, routes taken, and/or any other types of information. The stored information may be used for any purposes including, but not limited to, reporting, audits, etc.
4. Data report(s) database(s) 126. This database may store any data and/or data reports of any kind acquired and/or generated by the system 10.
5. Miscellaneous databases 128. These databases will store all other data required by the system 10.

It is understood that the above list of databases is meant for demonstration and that the system 10 may include some or all of the databases, and also may include additional databases as required. It is also understood that the scope of the system 10 is not limited in any way by the databases that it may include.

Various applications 104 and databases 102 in the system 10 may be accessible via interface(s) 142. These interfaces 142 may be provided in the form of APIs or the like and made accessible to external users T, AD via one or more gateways and interfaces 144 (e.g., via a web-based application 200 and/or a mobile application 200 running on a client's personal device 500 such as a mobile phone, tablet computer, desktop computer, laptop computer, etc.).

It is understood that any aspect and/or element of any embodiment described herein or otherwise may be combined in any way to form new embodiments all of which are easily understood by a person of ordinary skill in the art and all of which are within the scope of the system 10.

Those of ordinary skill in the art will appreciate and understand, upon reading this description, that embodiments hereof may provide different and/or other advantages, and that not all embodiments or implementations need have all advantages.

Computing

The services, mechanisms, operations, and acts shown and described above are implemented, at least in part, by software running on one or more computers or computer systems or devices. It should be appreciated that each user device is, or comprises, a computer system.

Programs that implement such methods (as well as other types of data) may be stored and transmitted using a variety of media (e.g., computer readable media) in a number of manners. Hard-wired circuitry or custom hardware may be used in place of, or in combination with, some or all of the software instructions that can implement the processes of various embodiments. Thus, various combinations of hardware and software may be used instead of software only.

One of ordinary skill in the art will readily appreciate and understand, upon reading this description, that the various processes described herein may be implemented by, e.g., appropriately programmed general purpose computers, special purpose computers and computing devices. One or more such computers or computing devices may be referred to as a computer system.

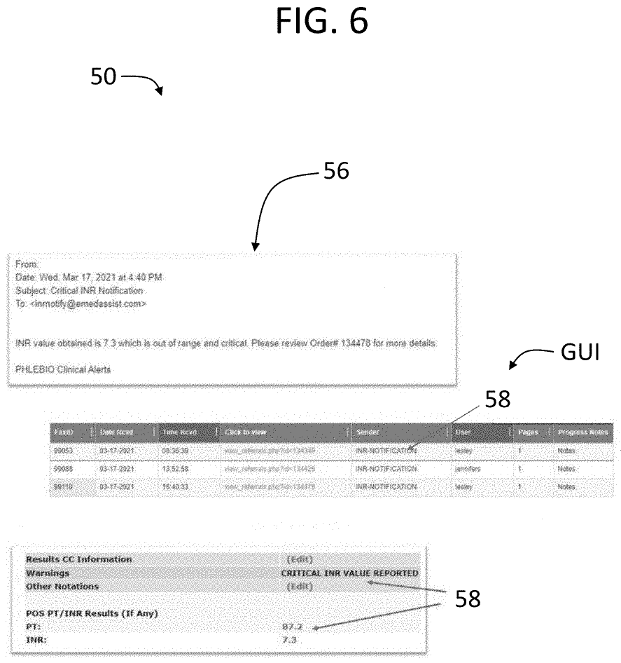

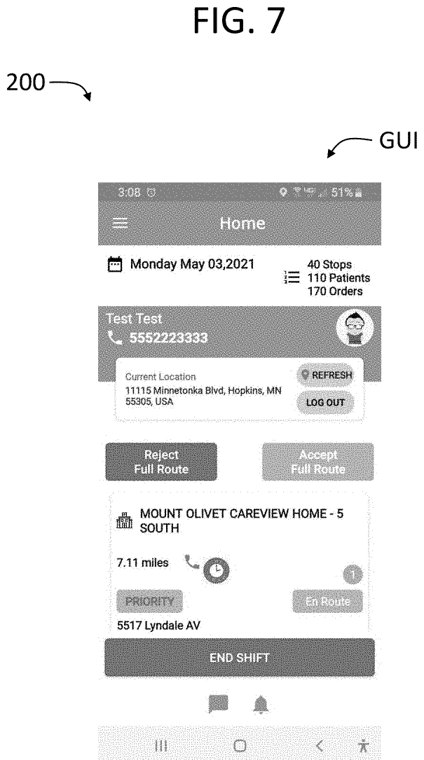

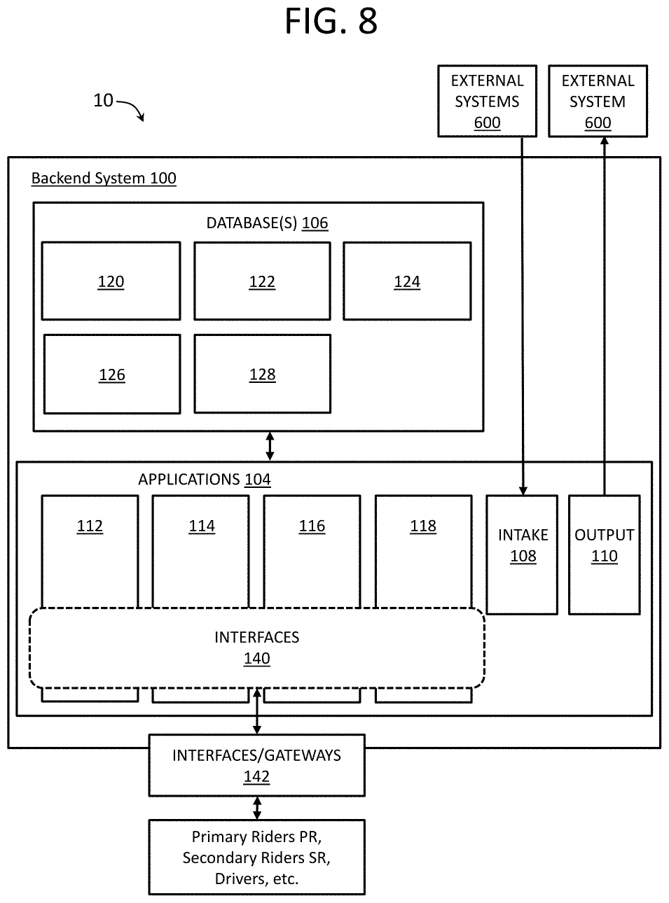

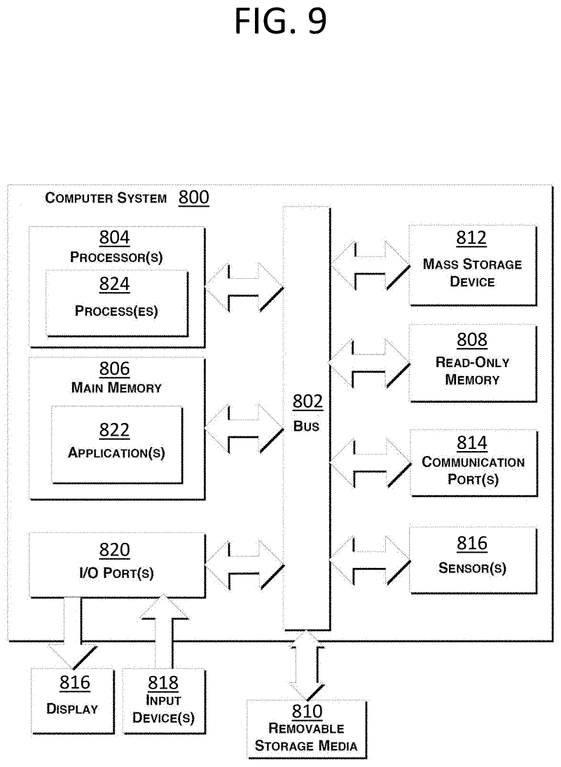

FIG. 6 is a schematic diagram of a computer system 800 upon which embodiments of the present disclosure may be implemented and carried out.

According to the present example, the computer system 800 includes a bus 802 (i.e., interconnect), one or more processors 804, one or more communications ports 814, a main memory 806, removable storage media 810, read-only memory 808, and a mass storage 812. Communication port(s) 814 may be connected to one or more networks by way of which the computer system 800 may receive and/or transmit data.

As used herein, a “processor” means one or more microprocessors, central processing units (CPUs), computing devices, microcontrollers, digital signal processors, or like devices or any combination thereof, regardless of their architecture. An apparatus that performs a process can include, e.g., a processor and those devices such as input devices and output devices that are appropriate to perform the process.

Processor(s) 804 can be (or include) any known processor, such as, but not limited to, an Intel® Itanium® or Itanium 2® processor(s), AMD® Opteron® or Athlon MP® processor(s), or Motorola® lines of processors, and the like.

Communications port(s) 814 can be any of an RS-232 port for use with a modem based dial-up connection, a 10/100 Ethernet port, a Gigabit port using copper or fiber, or a USB port, and the like. Communications port(s) 814 may be chosen depending on a network such as a Local Area Network (LAN), a Wide Area Network (WAN), a CDN, or any network to which the computer system 800 connects. The computer system 800 may be in communication with peripheral devices (e.g., display screen 816, input device(s) 818) via Input/Output (I/O) port 820. Some or all of the peripheral devices may be integrated into the computer system 800, and the input device(s) 818 may be integrated into the display screen 816 (e.g., in the case of a touch screen).

Main memory 806 can be Random Access Memory (RAM), or any other dynamic storage device(s) commonly known in the art. Read-only memory 808 can be any static storage device(s) such as Programmable Read-Only Memory (PROM) chips for storing static information such as instructions for processor(s) 804. Mass storage 812 can be used to store information and instructions. For example, hard disks such as the Adaptec® family of Small Computer Serial Interface (SCSI) drives, an optical disc, an array of disks such as Redundant Array of Independent Disks (RAID), such as the Adaptec® family of RAID drives, or any other mass storage devices may be used.

Bus 802 communicatively couples processor(s) 804 with the other memory, storage and communications blocks. Bus 802 can be a PCI/PCI-X, SCSI, a Universal Serial Bus (USB) based system bus (or other) depending on the storage devices used, and the like. Removable storage media 810 can be any kind of external hard-drives, floppy drives, IOMEGA® Zip Drives, Compact Disc-Read Only Memory (CD-ROM), Compact Disc-Re-Writable (CD-RW), Digital Versatile Disk-Read Only Memory (DVD-ROM), etc.

Embodiments herein may be provided as one or more computer program products, which may include a machine-readable medium having stored thereon instructions, which may be used to program a computer (or other electronic devices) to perform a process. As used herein, the term “machine-readable medium” refers to any medium, a plurality of the same, or a combination of different media, which participate in providing data (e.g., instructions, data structures) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random-access memory, which typically constitutes the main memory of the computer. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications.

The machine-readable medium may include, but is not limited to, floppy diskettes, optical discs, CD-ROMs, magneto-optical disks, ROMs, RAMs, erasable programmable read-only memories (EPROMs), electrically erasable programmable read-only memories (EEPROMs), magnetic or optical cards, flash memory, or other type of media/machine-readable medium suitable for storing electronic instructions. Moreover, embodiments herein may also be downloaded as a computer program product, wherein the program may be transferred from a remote computer to a requesting computer by way of data signals embodied in a carrier wave or other propagation medium via a communication link (e.g., modem or network connection).

Various forms of computer readable media may be involved in carrying data (e.g. sequences of instructions) to a processor. For example, data may be (i) delivered from RAM to a processor; (ii) carried over a wireless transmission medium; (iii) formatted and/or transmitted according to numerous formats, standards or protocols; and/or (iv) encrypted in any of a variety of ways well known in the art.

A computer-readable medium can store (in any appropriate format) those program elements that are appropriate to perform the methods.

As shown, main memory 806 is encoded with application(s) 822 that support(s) the functionality as discussed herein (an application 822 may be an application that provides some or all of the functionality of one or more of the mechanisms described herein). Application(s) 822 (and/or other resources as described herein) can be embodied as software code such as data and/or logic instructions (e.g., code stored in the memory or on another computer readable medium such as a disk) that supports processing functionality according to different embodiments described herein.

During operation of one embodiment, processor(s) 804 accesses main memory 806 via the use of bus 802 in order to launch, run, execute, interpret or otherwise perform the logic instructions of the application(s) 522. Execution of application(s) 822 produces processing functionality of the service(s) or mechanism(s) related to the application(s). In other words, the process(es) 824 represents one or more portions of the application(s) 822 performing within or upon the processor(s) 804 in the computer system 800.

It should be noted that, in addition to the process(es) 824 that carries(carry) out operations as discussed herein, other embodiments herein include the application 822 itself (i.e., the un-executed or non-performing logic instructions and/or data). The application 822 may be stored on a computer readable medium (e.g., a repository) such as a disk or in an optical medium. According to other embodiments, the application 822 can also be stored in a memory type system such as in firmware, read only memory (ROM), or, as in this example, as executable code within the main memory 806 (e.g., within Random Access Memory or RAM). For example, application 822 may also be stored in removable storage media 810, read-only memory 808, and/or mass storage device 812.

Those skilled in the art will understand that the computer system 800 can include other processes and/or software and hardware components, such as an operating system that controls allocation and use of hardware resources.

As discussed herein, embodiments of the present invention include various steps or operations. A variety of these steps may be performed by hardware components or may be embodied in machine-executable instructions, which may be used to cause a general-purpose or special-purpose processor programmed with the instructions to perform the operations. Alternatively, the steps may be performed by a combination of hardware, software, and/or firmware. The term “module” refers to a self-contained functional component, which can include hardware, software, firmware or any combination thereof.

One of ordinary skill in the art will readily appreciate and understand, upon reading this description, that embodiments of an apparatus may include a computer/computing device operable to perform some (but not necessarily all) of the described process.

Embodiments of a computer-readable medium storing a program or data structure include a computer-readable medium storing a program that, when executed, can cause a processor to perform some (but not necessarily all) of the described process.

Where a process is described herein, those of ordinary skill in the art will appreciate that the process may operate without any user intervention. In another embodiment, the process includes some human intervention (e.g., a step is performed by or with the assistance of a human).

As used in this description, the term “portion” means some or all. So, for example, “A portion of X” may include some of “X” or all of “X”. In the context of a conversation, the term “portion” means some or all of the conversation.

As used herein, including in the claims, the phrase “at least some” means “one or more,” and includes the case of only one. Thus, e.g., the phrase “at least some ABCs” means “one or more ABCs”, and includes the case of only one ABC.

As used herein, including in the claims, the phrase “based on” means “based in part on” or “based, at least in part, on,” and is not exclusive. Thus, e.g., the phrase “based on factor X” means “based in part on factor X” or “based, at least in part, on factor X.” Unless specifically stated by use of the word “only”, the phrase “based on X” does not mean “based only on X.”

As used herein, including in the claims, the phrase “using” means “using at least,” and is not exclusive. Thus, e.g., the phrase “using X” means “using at least X.” Unless specifically stated by use of the word “only”, the phrase “using X” does not mean “using only X.”

In general, as used herein, including in the claims, unless the word “only” is specifically used in a phrase, it should not be read into that phrase.

As used herein, including in the claims, the phrase “distinct” means “at least partially distinct.” Unless specifically stated, distinct does not mean fully distinct. Thus, e.g., the phrase, “X is distinct from Y” means that “X is at least partially distinct from Y,” and does not mean that “X is fully distinct from Y.” Thus, as used herein, including in the claims, the phrase “X is distinct from Y” means that X differs from Y in at least some way.

As used herein, including in the claims, a list may include only one item, and, unless otherwise stated, a list of multiple items need not be ordered in any particular manner. A list may include duplicate items. For example, as used herein, the phrase “a list of XYZs” may include one or more “XYZs”.

It should be appreciated that the words “first” and “second” in the description and claims are used to distinguish or identify, and not to show a serial or numerical limitation. Similarly, the use of letter or numerical labels (such as “(a)”, “(b)”, and the like) are used to help distinguish and/or identify, and not to show any serial or numerical limitation or ordering.

No ordering is implied by any of the labeled boxes in any of the flow diagrams unless specifically shown and stated. When disconnected boxes are shown in a diagram the activities associated with those boxes may be performed in any order, including fully or partially in parallel.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

1. A method of providing mobile medical services, the method comprising:
(A) receiving a first workorder for a first medical procedure to be performed on a first patient at a first location, wherein the first medical procedure includes acquiring at least one medical specimen from the first patient;
(B) providing one or more mobile medical vehicles;
(C) assigning the first workorder to a first one of the one or more mobile medical vehicles;
(D) providing, on a graphical use interface (GUI), the first route to the first one of the one or more mobile medical vehicles;
(E) providing, on a graphical user interface (GUI), guidance for taking the at least one medical specimen from the first patient;
(F) performing, with a medical test meter, at least one diagnostic test on the at least one medical specimen;
(G) receiving, from the medical test meter, first diagnostic data regarding the at least one medical specimen;
(H) determining if the first diagnostic data is outside a defined normal diagnostic data range;
(I) upon a determination that the first diagnostic data is outside the defined normal diagnostic range, then:
(J) communicating the first diagnostic data to a first recipient; and
(K) causing a medical treatment for the first user to be tailored based at least in part on the first diagnostic data.

2. The method of claim 1 wherein the first medical procedure includes a phlebotomy procedure.

3. The method of claim 1 wherein the first medical procedure includes at least one of a prothrombin time (PT) test, a partial thromboplastin time (PTT) test, and an Internationalized Normalized Ratio (INR) test.

4. The method of claim 1 wherein the medical test meter includes at least one of a prothrombin time (PT) test meter, a partial thromboplastin time (PTT) test meter, and an Internationalized Normalized Ratio (INR) test meter.

5. The method of claim 1 wherein the assigning the first workorder to a first one of the one or more mobile medical vehicles in (C) further includes:
(C)(1) for each one of the one or more mobile medical vehicles, determining a route between the each one of the one or more mobile medical vehicles and the first location;
(C)(2) based at least in part on the route determined for each one of the one or more mobile medical vehicles in (C)(1), determining the first one of the one or more medical vehicles to be assigned the first workorder.

6. The method of claim 1 further comprising:
(L) using a navigation system, determining a first distance traveled by the first one of the one or more mobile medical vehicles to the first location;
(M) based at least in part on the first distance traveled, determining a first travel charge to be provided to a first insurance provider for the first patient.

7. The method of claim 1 further comprising:
(L) determining if the at least one medical specimen includes two or more medical specimens;
(M) determining if each of the two or more medical specimens are available from the first patient;
(N) upon a determination that at least one of the two or more medical specimens are not available from the first patient, then:
(O) scheduling a second medical procedure to be performed on the first patient at the first location, wherein the second medical procedure includes the taking of the at least one of the two or more medical specimens not available.

8. The method of claim 1 further comprising:
(J)(1) highlighting the first diagnostic data.

9. The method of claim 8 wherein the highlighting the first diagnostic data includes designating the first diagnostic data as outside the defined normal diagnostic range.

10. The method of claim 1 wherein the causing a medical treatment for the first user to be tailored based at least in part on the first diagnostic data in (K) further comprises:
(K)(1) recommending to the first recipient a prescription dosage of a first medication to be prescribed to the first patient.

11. The method of claim 10 wherein the first recipient includes at least one of a healthcare provider, a physician, a nurse, a hospital, a medical office, and an administrator.

12. The method of claim 1 wherein the guidance for taking the at least one medical specimen from the first patient provided in (E) includes guidance on medical equipment required for the first medical procedure.

13. A method of providing mobile medical services, the method comprising:
(A) receiving a first workorder for a first medical procedure to be performed on a first patient at a first location, wherein the first medical procedure includes acquiring at least one medical specimen from the first patient;
(B) providing one or more mobile medical vehicles;
(C) assigning the first workorder to a first one of the one or more mobile medical vehicles including at least one technician;
(D) providing, on a graphical use interface (GUI), the first route to the first one of the one or more mobile medical vehicles;
(E) providing to the at least one technician, on a graphical user interface (GUI), guidance for taking the at least one medical specimen from the first patient;
(F) performing, with a medical test meter, at least one diagnostic test on the at least one medical specimen;
(G) receiving, from the medical test meter, first diagnostic data regarding the at least one medical specimen;
(H) determining if the first diagnostic data is outside a defined normal diagnostic data range;
(I) upon a determination that the first diagnostic data is outside the defined normal diagnostic range, then:
(J) designating the first diagnostic data as outside the defined normal diagnostic range;
(K) communicating the first diagnostic data to a first recipient; and
(L) causing a medical treatment for the first user to be tailored based at least in part on the first diagnostic data;
(M) upon a determination that the first diagnostic data is not outside the defined normal diagnostic range, then:
(N) designating the first diagnostic data as not outside the defined normal diagnostic range; and
(O) communicating the first diagnostic data to a first recipient.

14. The method of claim 13 wherein the first medical procedure includes a phlebotomy procedure.

15. The method of claim 13 wherein the first medical procedure includes at least one of a prothrombin time (PT) test, a partial thromboplastin time (PTT) test, and an Internationalized Normalized Ratio (INR) test.

16. The method of claim 13 wherein the medical test meter includes at least one of a prothrombin time (PT) test meter, a partial thromboplastin time (PTT) test meter, and an Internationalized Normalized Ratio (INR) test meter.

17. The method of claim 1 wherein the assigning the first workorder to a first one of the one or more mobile medical vehicles in (C) further includes:
(C)(1) for each one of the one or more mobile medical vehicles, determining a route between the each one of the one or more mobile medical vehicles and the first location;
(C)(2) based at least in part on the route determined for each one of the one or more mobile medical vehicles in (C)(1), determining the first one of the one or more medical vehicles to be assigned the first workorder.

18. The method of claim 1 further comprising:
(P) using a navigation system, determining a first distance traveled by the first one of the one or more mobile medical vehicles to the first location;
(Q) based at least in part on the first distance traveled, determining a first travel charge to be provided to a first insurance provider for the first patient.

19. The method of claim 1 wherein the causing a medical treatment for the first user to be tailored based at least in part on the first diagnostic data in (L) further comprises:
(L)(1) recommending to the first recipient a prescription dosage of a first medication to be prescribed to the first patient.

20. The method of claim 1 further comprising:
(P) determining if the at least one medical specimen includes two or more medical specimens;
(Q) determining if each of the two or more medical specimens are available from the first patient;
(R) upon a determination that at least one of the two or more medical specimens are not available from the first patient, then:
(S) scheduling a second medical procedure to be performed on the first patient at the first location, wherein the second medical procedure includes the taking of the at least one of the two or more medical specimens not available.
